(19) United States
(12) Patent Application Publication
(10) Pub. No.: US 2005/0163652 A1
(43) Pub. Date: Jul. 28, 2005

(54) Low level ultraviolet disinfecting system

(76) Inventors: Richard N. Metzger, Pasadena, CA (US); Keith M. Crawford, Valencia, CA (US); David L. Witham, Ventura, CA (US)

Correspondence Address: CISLO & THOMAS, LLP, 233 WILSHIRE BLVD, SUITE 900, SANTA MONICA, CA, 90401-1211, US

(21) Appl. No.: 11/033,659

(22) Filed: Jan. 11, 2005

Related U.S. Application Data
(60) Provisional application No. 60/538,641, filed on Jan. 23, 2004.

(51) Int. Cl. A61L 9/20; A61L 9/18

(52) U.S. Cl. 422/22; 422/121; 422/24

(57) ABSTRACT

A disinfecting system comprises a light source having output suitable for use as a germicidal agent, and a power supply for the light source that is adapted to limit the output of the light source to levels adequate for microbial growth control. The light source is operatively housed in fluid-conveying equipment for disinfecting fluids and surfaces therein. The limited output is attenuated by fluid-conveying equipment components disposed within the output range of the light source. The attenuated output provides safe human exposure levels in the vicinity of fluid intake and exhaust portions of the fluid-conveying equipment. The limited output inhibits the degradation of fluid-conveying equipment components disposed within the output range of the light source. The light source may be covered with a thin film or sleeve of material being semi-transparent to germicidal UV wavelengths to control the output of the light source.

### CROSS-REFERENCE TO RELATED APPLICATION

This application claims priority to U.S. Provisional Application No. 60/538,641, filed on Jan. 23, 2004, the entire contents of which are hereby incorporated by reference.

### BACKGROUND

Use of ultra violet (UV) energy in HVAC (Heating Ventilation and Air Conditioning) systems is becoming very common. UV energy is beneficial to the HVAC system in that it is effective in reducing the spread of undesirable microorganisms such as molds, bacteria and viruses on surfaces and within media, such as air or water or other fluids. UV energy is also effective in maintaining surfaces free of biological growth over a period of time, where the surface is regularly exposed to UV energy. Even in low dosages, UV energy is effective in is maintaining surfaces free of biological growth with regular exposure.

While the use of UV energy has proven beneficial, there are known risks and negative aspects of its use. Certain materials may degrade rapidly from UV exposure. Many of these materials may be used within an HVAC system such as insulation, gaskets and electrical insulation, among others. UV energy, particularly in the germicidal range (approximately 254 nanometers) may be harmful to the skin and eyes of humans and animals. Guidelines for acceptable levels of human exposure are established and published by NIOSH (National Institute of Occupational Safety and Health) and the ACGIH (American Conference of Governmental Industrial Hygienists).

Recently, many companies utiliting UV energy for disinfection purposes within HVAC system have been promoting higher and higher levels of UV energy and higher output UV lamps. High UV levels and dosages, and short exposure times may be utilized, for example, in disinfecting a moving air stream. However, these levels may not be required when irradiating surfaces over long periods of time, such as air conditioning coils and drain pans. In these types of applications, excessively high levels of UV may damage components and may require shielding of certain materials and components. Furthermore, precautions must be taken for human exposure.

In a typical “in-duct” system or large system as shown in FIG. 1, there is relatively little danger of human exposure as the UV lamps are disposed far from, and on irregular paths from, any intake or exhaust grills, where there may be human exposure. In configurations such as these, only system components may need to be shielded, and higher output UV lamps may be used.

When UV energy is utilized in air conditioning systems, where the lamps are left operating for long periods of time, lower levels of UV energy emissions may be sufficient.

Another problem may arise when UV lamps are operated at lower levels than the current at which UV lamps are rated. This situation may cause the lamp to fail prematurely, which may lead to increased operating costs of the system. What is needed is a disinfecting system that may operate at lower energy levels while maintaining reliability and lower operating costs.

In many small HVAC systems, there is very limited space for mounting the UV lamp. It may be extremely difficult to fit the UV lamp within an existing HVAC installation. Partial disassembly of the HVAC system is often required in order to access the space where UV lamp support brackets must be mounted. What is also needed is an easily installed mechanical mechanism to support the UV lamp within the HVAC system.

### SUMMARY

Exemplary embodiments disclosed herein are directed to a low level UV disinfecting system and method.

In accordance with one aspect of the invention, a disinfecting system comprises at least one light source having output suitable for use as a germicidal agent, and means for limiting the output of said at least one light source to levels adequate for microbial growth control. The light source is operatively housed in fluid-conveying equipment for fluid disinfection whether the fluid be air, water, or other fluid.

In accordance with another aspect of the invention, a disinfecting method comprises the steps of providing at least one light source having output suitable for use as a germicidal agent, limiting the output of the light source to levels adequate for microbial growth control, and attenuating the limited output by fluid-conveying equipment components disposed within the output range of the light source. The light source is operatively housed in fluid-conveying equipment for fluid disinfection. The attenuated output provides safe human exposure levels in the vicinity of fluid intake and exhaust portions of the fluid-conveying equipment.

In accordance with yet another aspect of the invention, a disinfecting system comprises an electromagnetic energy source, and a power supply designed to supply current to the electromagnetic energy source such that a lower level of energy than rated by a standard energy source is emitted, by using a near rated cathode current to maintain lamp life, and a percentage of rated lamp current, within HVAC equipment. The energy output is sufficiently controlled to reduce microbial growth and energy output is reduced to prohibit unsafe levels and reduce degradation of HVAC system components.

In accordance with still another aspect of the invention, a method of disinfecting air, surfaces, fluids and other things utilizing a lower than standard or rated level of electromagnetic energy comprises the steps of providing an electromagnetic energy source, electrically coupling a power supply to the electromagnetic energy source, and powering the electromagnetic energy source with a percentage of rated lamp current, and near rated cathode current to reduce emitted energy and to enhance the life of the electromagnetic energy source.

In accordance with a further aspect of the invention, a disinfecting system for disinfecting air, surfaces, fluids and other objects comprises an electromagnetic energy source, and a power supply electrically coupled to the electromagnetic energy source. The electromagnetic energy is emitted at lower than rated levels by supplying the electromagnetic energy source with near rated cathode current and a percentage of rated lamp current. The emitted energy is sufficiently controlled to reduce microbial growth and otherwise disinfect, and the emitted energy is reduced to prohibit unsafe levels of emitted energy and to reduce degradation of system components.

In accordance with a still further aspect of the invention, a method of supplying lower than rated levels of electromagnetic energy utilizing a rated electromagnetic energy source comprises supplying the electromagnetic energy source with near rated, cathode current to maintain cathode temperature to allow for acceptable thermal characteristics of the electromagnetic energy source, and supplying an electromagnetic energy source with a fraction of rated, lamp current to allow the electromagnetic energy source to operate at below rated levels and emit lower than rated or maximum levels of electromagnetic energy.

In accordance with yet further aspect of the invention, a disinfecting system for disinfecting air, surfaces, fluids and other objects comprises an electromagnetic energy source, and a power supply electrically coupled to the electromagnetic energy source. The electromagnetic energy is emitted at lower than rated levels by supplying the electromagnetic energy source with near rated cathode current and a percentage of rated lamp current.

These and other aspects of the invention will become apparent from a review of the accompanying drawings and the following detailed description of the invention.

### BRIEF DESCRIPTION OF THE DRAWINGS

The invention is generally shown by way of reference to the accompanying drawings in which:

FIG. 1 is a cross-sectional view of UV energy utilization in an in-duct HVAC system.

FIG. 2 is a cross-sectional view of a small to medium, self-contained, HVAC system with a UV energy source in accordance with an exemplary embodiment of the present invention.

FIG. 3 is an isometric view of a representative HVAC system with a UV energy source in accordance with an exemplary embodiment of the present invention.

FIG. 4 is block diagram of an electric circuit in accordance with an exemplary embodiment of the present invention.

FIG. 5 is a side perspective view of a support clip for a UV energy source in accordance with an exemplary embodiment of the present invention.

### DETAILED DESCRIPTION

The detailed description set forth below in connection with the appended drawings is intended as a description of exemplary embodiments and is not intended to represent the only forms in which the exemplary embodiments may be constructed and/or utilized. The description sets forth the functions and the sequence of steps for constructing and operating the exemplary embodiments in connection with the illustrated embodiments. However, it is to be understood that the same or equivalent functions and sequences may be accomplished by different embodiments that are also intended to be encompassed within the spirit and scope of the invention.

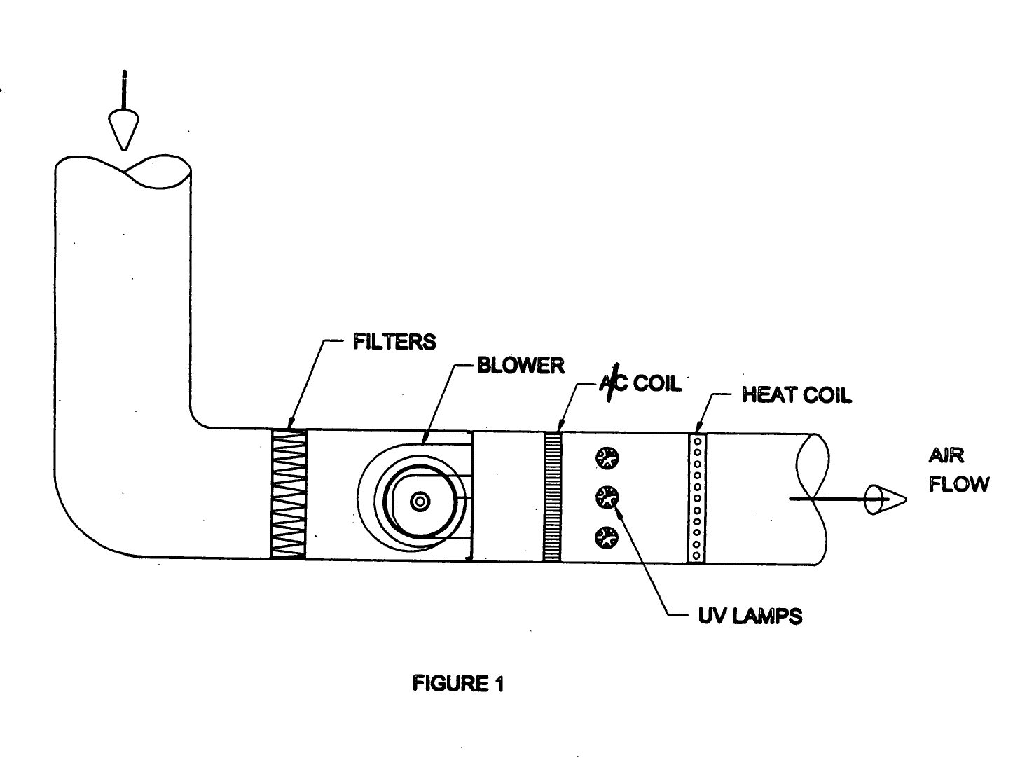

Some embodiments of the invention will be described in detail with reference to the related drawings of FIGS. 1-5. Additional embodiments, features and/or advantages of the invention will become apparent from the ensuing description or may be learned by practicing the invention. In the figures, the drawings are not to scale with like numerals referring to like features throughout both the drawings and the description.

FIG. 1 is a cross-sectional view of an in-duct disinfecting system. The system includes filters, blower, A/C (Air Conditioning) coil, and a heating coil. The disinfecting system also includes ultraviolet lamps or other electromagnetic energy sources for disinfecting surfaces, air, fluid, and other things, as desired. This in-duct disinfecting system may employ high-output UV lamps as the UV lamps are typically disposed far from, and on irregular paths from any intake or exhaust grills, i.e. there is relatively little risk of human exposure.

Air may enter the system and pass through the filters, the blower and through the A/C coil to cool the air as well as remove moisture from the air. The UV lamps, which are disposed downstream of the A/C coil, disinfect the air stream, whereby the air stream may pass through the heating coil, if needed. The UV lamps disinfect the A/C coil, the heating coil as well as other system surfaces, fluids, and objects, as desired.

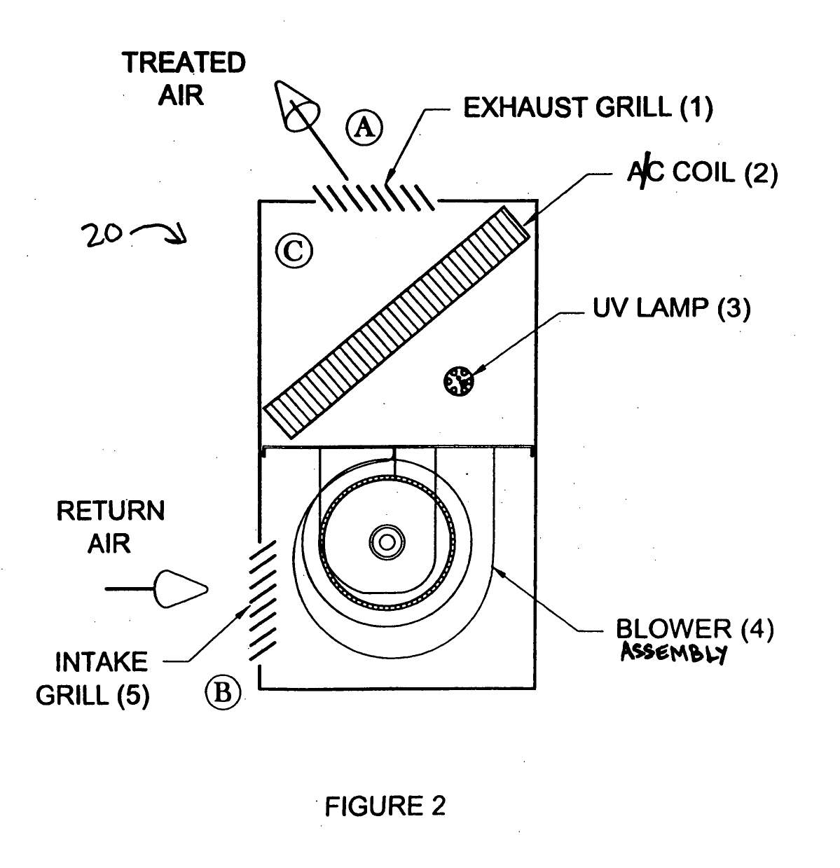

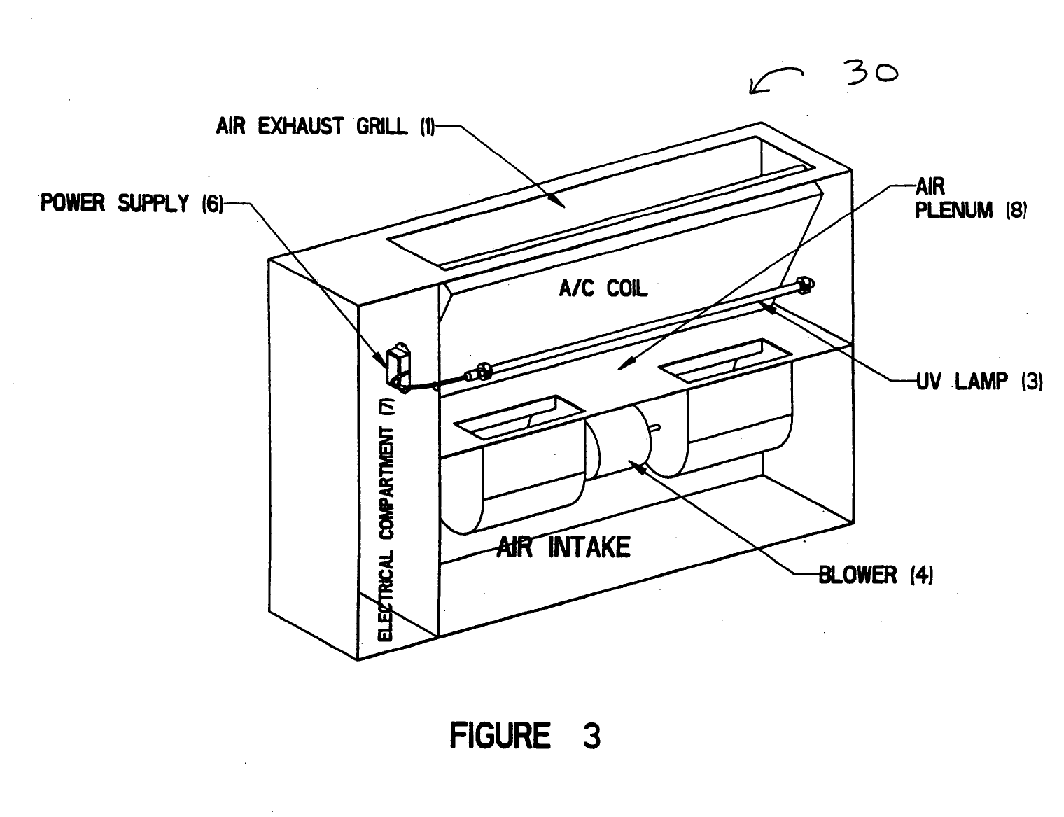

Exemplary embodiments may involve generally small to medium, self-contained HVAC systems, as shown, for example, in FIGS. 2 and 3. These systems may be in the size range of 1 to 10 tons of air conditioning, and may include, but are not limited to, fan coil units, window units, PTAC (Packaged Terminal Air Conditioning) units, heat pumps, unit ventilators and above ceiling mounted units, among others. This type of system may be used in hotels, motels, offices, homes, etc. Due to the geometry of the equipment and limited space available, the electromagnetic energy (light) source, such as a UV lamp, must often be applied upstream of the A/C coil. The A/C coils (fin sections) may be less deep in these smaller systems than in large systems and often only have approximately 2 rows of coils.

Referring to FIG. 2, if large amounts of UV energy are utilized, the amount of energy passing through the coils and exiting from the exhaust grill may exceed safe human exposure levels. Furthermore, if the lamps are positioned on the downstream side of the A/C coil, in the vicinity of area “C”, excessive UV energy (exceeding safe human exposure levels) may also occur in the vicinity of area “A”. A typical standard output UV lamp employed on the upstream side of the A/C coil may generate enough UV energy to cause significant amounts of UVC energy to emit through the two-row A/C coil and into the UV sensitive areas. Mechanical or optical baffles may be installed near area “C” and area “B” to reduce the amount of UV energy exiting the grills. However, these baffles may result in reduced airflow from the unit and/or increase the pressure drop that the HVAC blower must overcome. These methods of attenuating the UV energy leaving the grill are complex and costly.

In accordance with the general principles of this invention, UV energy levels may be reduced via the power supply for the UV lamp. Specifically, UV energy output may be limited to levels adequate for microbial growth control and yet a level where normal attenuation of UV energy by systems components, such as the coil and grills, keeps UV energy levels in the vicinity of areas “A” and “B” (FIG. 2) within safe levels.

FIG. 2 shows a cross-sectional view of a HVAC unit including an exemplary embodiment of the system. The electromagnetic energy source or UV lamp 3 may be mounted within the unit using mounting clips or simple attachment hardware. UV lamp output is controlled via the lamp power supply (not shown). Specifically, the UV lamp output is lowered to levels being adequate for microbial growth control. HVAC components, including but not limited to A/C coil 2, exhaust grill 1, blower assembly 4, and intake grill 5 attenuate some of the UV energy output by the lamp. As a result, UV energy output levels in the vicinity of areas “A” & “B” are kept at safe levels.

FIG. 2 shows a cross-sectional view of an exemplary embodiment of a disinfecting system, generally at 20. Disinfecting system 20 comprises an exhaust grill 1 that may be configured to allow air to pass from area “C” (interior) to area “A” (exterior), as generally shown by the fluid flow arrow in FIG. 2. Disinfecting system 20 also comprises an A/C coil 2, an electromagnetic energy (light) source or UV lamp 3, and power supply (not shown) for the UV lamp. UV lamp 3 disinfects system surfaces, fluids, air, and other things, as desired. UV lamp 3 may be operated at lower than rated current levels to emit lower than maximum or rated UV or electromagnetic energy, as desired. If the electromagnetic energy emitted is at lower levels for long periods of time, it disinfects the system as well as a UV lamp being operated at full power and emitting the full rated amount of electromagnetic energy. With the lower than rated emission, the UV lamp may disinfect as well, while not degrading the other components of the system as rapidly, as well as not causing excess electromagnetic radiation to exit the system via exhaust grill 1, or other part of the system, and possibly harming persons, animals or other objects outside the system.

This system may also include a blower 4 configured to move air through the unit from area B through intake grill 5 by UV lamp 3 and A/C coil 2 and exit exhaust grill 1 in the vicinity of area A. With this configuration, smaller air handling units may utilize a lower level UV disinfecting system to allow the components of the system to not degrade as rapidly as when a full power UV system is utilized. Furthermore, if UV lamp 3 is operated in accordance with the general principles of the present invention, UV lamp life may be enhanced or extended, such that the lamp will not have to be replaced as often, thereby reducing the overall cost of operating the system. In addition, operating the UV lamp at lower power levels may reduce the electrical cost of operating the system.

FIG. 3 is a cross-sectional view of a disinfecting system 30, according to another exemplary embodiment. Disinfecting system 30 comprises an air exhaust grill 1 which allows air to pass therethrough from the interior of the system to the exterior of the system. Disinfecting system 30 also comprises an electromagnetic energy (light) source, or UV lamp 3 for disinfecting air, fluid, surfaces, and other things, as desired. Blower 4 moves air from “AIR IN-TAKE” to air exhaust grill 1 via the A/C coil and around UV lamp 3.

Disinfecting system 30 also comprises a power supply 6 which supplies power to UV lamp 3. Power supply 6 is operatively coupled to UV lamp 3. Power supply 6 may be configured to provide sufficient cathode current to UV lamp 3 to keep the cathodes at a certain temperature such that the life of UV lamp 3 is enhanced and/or extended. The supplied cathode current maintains the ‘hot spot’ temperature at the cathode to facilitate the plasma arc within the UV lamp. This ‘hot spot’ temperature is critical to a long term reliable operation of the cathode. Furthermore, power supply 6 may provide a lamp current that is a fraction of the current rated for the lamp. In this manner, a fraction of the UV energy output is emitted from the UV lamp, while maintaining the life expectancy of the lamp. Furthermore, the lower level UV energy emissions cause less degradation of system components with the emissions being generally safer in regard to human or animal exposure. This lower level emitted from the system may protect people and other things outside the system from being exposed to harmful levels of electromagnetic radiation generated by disinfecting system 30.

In FIG. 3, another exemplary embodiment is shown in a fan coil unit. Power supply 6 is mounted in an electrical compartment 7, i.e. remote from air plenum 8. Power supply 6 may also be mounted within the air-moving portion of the system, or at other locations, as desired.

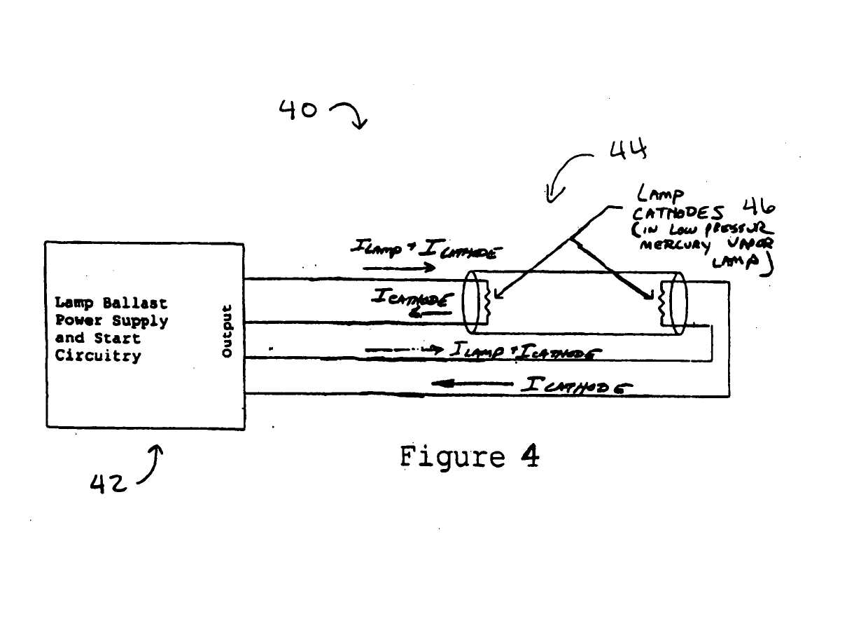

FIG. 4 is a block diagram of an electric circuit 40 according to an exemplary embodiment. Circuit 40 includes a lamp ballast, power supply and start circuitry 42 adapted to supply a cathode current, I_{cathode}, and a lamp current, Iₗₐₘₚ. The cathode current maintains a certain temperature at the cathodes to allow for acceptable thermal emission from light source 44. If lamp current, Iₗₐₘₚ, is provided at a fraction of the rated current of electromagnetic energy source 44, then less than maximum electromagnetic energy would be emitted by light source 44. In this manner, lower levels of electromagnetic energy may be emitted from a standard, rated light source or UV lamp. In addition, operation of the light source in this manner provides a reduction in overall power consumption. This results in lower operating costs than with a traditional lamp operated at full power.

Light source 44 may be a low-pressure mercury vapor (LPMV) lamp, UV energy source, or other electromagnetic energy source, as desired. The lamp ballast may be configured to electrically couple to current and future electromagnetic energy sources. Furthermore, the lamp ballast may also be configured to physically fit with other current and future lamp systems, as well as other systems, as desired. Alternatively, the lamp ballast and light source 44 may be made integral with each other such that they may be replaced and/or added to a new or existing system together as one unit. System 30, as generally shown in FIG. 3, may be a small type air-handling unit such as a window air conditioner or other air-handling unit in the range of up to 20 tons, or larger, as desired.

In this exemplary embodiment, the power source (ballast) driving the LPMV lamp provides a lamp current (Iₗₐₘₚ) that may be a fraction of the nominal or rated lamp current. In addition, the power source driving the lamp provides sufficient cathode current (I_{cathode}) to maintain the ‘hot spot’ temperature at the cathode to facilitate the plasma arc within the LPMV lamp. This ‘hot spot’ temperature is critical to a long-term reliable operation of the LPMV lamp cathode. Failure to provide such energy to the cathodes of a LPMV lamp is a primary cause for premature catastrophic failure of LPMV lamps, and/or UVC energy output maintenance over the operational life of the lamp.

In FIG. 4, the ballast power supply provides two critical components of energy to a low-pressure mercury vapor lamp. The first component is the lamp current (Iₗₐₘₚ), which flows from a first cathode to a second cathode through the lamp gas plasma. This lamp current is the main source of energy that excites the lamp gas to provide UV light. In this invention, Iₗₐₘₚ is preferably set at a fraction of the nominal or rated lamp current specified for the lamp.

The second component of energy is the cathode current (I_{cathode}), which circulates through each of the lamp cathodes. This cathode current serves to maintain adequate heat in the cathodes to allow for reliable thermal emission of the lamp. When operating the lamp at lamp current significantly below the nominal rated lamp current, this cathode current is necessary to ensure adequate reliability and stable operation of the lamp. Operation at lamp currents significantly under the nominal rating for the lamp without the addition of adequate supplementary cathode current may result in unstable lamp performance and significant lamp life reduction.

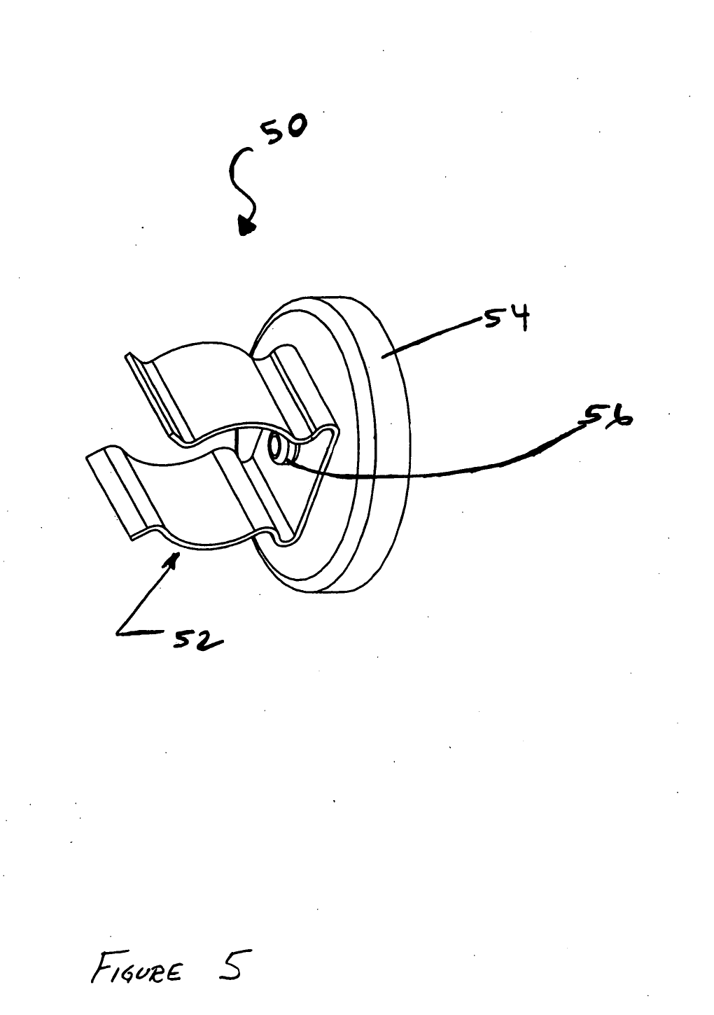

The UV light source of the present invention may be readily mounted in a space within a HVAC system where access with conventional tools is difficult or even impossible using a magnetic support clip 50, as generally shown in FIG. 5. Magnetic support clip 50 comprises a spring bracket 52 attached to a permanent magnet base 54 via a screw or rivet 56. Magnetic support clip 50 is intended to be used in pairs with spring bracket 52 being adapted to removably support each end of a UV lamp.

In accordance with another exemplary embodiment of the present invention, the UV lamp may be covered with a thin film (coating) or sleeve of material that is semi-transparent to germicidal UV wavelengths. The coating or sleeve being applied should be sufficiently thick to produce the desired UVC transmittance of the lamp. The material may have WVC transmittance in the range of about 10% to 95%. Applying such a thin film or sleeve shrunk to the UV lamp in accordance with the general principles of the present invention would effectively lower the UV energy output of the lamp to levels sufficient to control and/or reduce microbial growth, and prevent the undesired degradation of HVAC system components disposed within the output range of the UV lamp. Consequently, there would be no need to adapt a standard UV lamp power supply to limit the output of the UV lamp, as described generally hereinabove.

In closing, it is to be understood that the exemplary embodiments described herein are illustrative of the principles of the present invention. Other modifications that may be employed are within the scope of the invention. Thus, by way of example, but not of limitation, alternative configurations may be utilized in accordance with the teachings herein. Accordingly, the drawings and description are illustrative and not meant to be a limitation thereof. Moreover, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Thus, it is intended that the invention cover all embodiments and variations thereof as long as such embodiments and variations come within the scope of the appended claims and their equivalents.

1. A disinfecting system comprising:
at least one light source having output suitable for use as a germicidal agent, said at least one light source being operatively housed in fluid-conveying equipment for fluid disinfection; and
means for limiting the output of said at least one light source to levels adequate for microbial growth control, said limited output being attenuated by fluid-conveying equipment components disposed within the output range of said at least one light source, said attenuated output providing safe human exposure levels in the vicinity of fluid intake and exhaust portions of the fluid-conveying equipment.

2. The disinfecting system of claim 1, wherein said at least one light source is adapted to emit UVC (Ultraviolet C) energy.

3. The disinfecting system of claim 1, wherein the fluid-conveying equipment is HVAC (Heating, Ventilation, and Air Conditioning) equipment.

4. The disinfecting system of claim 1, wherein said output limiting means includes at least one power supply adapted to control the output of said at least one light source, said controlled output being limited to levels adequate for microbial growth control.

5. The disinfecting system of claim 4, wherein said at least one light source is powered by said at least one power supply.

6. The disinfecting system of claim 5, wherein said at least one power supply is mounted within a fluid-moving portion of the fluid-conveying equipment.

7. The disinfecting system of claim 5, wherein said at least one power supply is mounted away from a fluid-moving portion of the fluid-conveying equipment.

8. The disinfecting system of claim 7, wherein said at least one light source is mounted upstream of an A/C (Air Conditioning) coil.

9. The disinfecting system of claim 3, wherein said HVAC equipment includes at least one PTAC (Packaged Terminal Air Conditioning) unit.

10. The disinfecting system of claim 3, wherein said HVAC equipment includes at least one fan coil unit.

11. The disinfecting system of claim 3, wherein said HVAC equipment includes at least one window-mounted air conditioning unit.

12. The disinfecting system of claim 3, wherein said HVAC equipment includes at least one heat pump.

13. The disinfecting system of claim 3, wherein said HVAC equipment includes at least one unit ventilator.

14. The disinfecting system of claim 3, wherein said HVAC equipment includes at least one above ceiling-mounted air conditioning unit.

15. The disinfecting system of claim 3, wherein said HVAC equipment includes at least one air conditioning unit in the range of about 1 to 10 tons.

16. The disinfecting system of claim 8, wherein said at least one light source is mounted to an interior surface of the fluid-conveying equipment via mounting clips.

17. The disinfecting system of claim 16, wherein each of said mounting clips comprises a spring bracket attached to a magnet base.

18. The disinfecting system of claim 17, wherein said spring bracket is being attached to said magnet base using at least one screw.

19. The disinfecting system of claim 17, wherein said spring bracket is being attached to said magnet base using at least one rivet.

20. The disinfecting system of claim 1, wherein said output limiting means includes at least one film of material being semi-transparent to germicidal UV wavelengths and adapted to cover said at least one light source to control the output of said at least one light source, said controlled output being limited to levels adequate for microbial growth control.

21. The disinfecting system of claim 20, wherein said at least one film of material has UVC transmittance in the range of about 10% to 95%.

22. The disinfecting system of claim 1, wherein said output limiting means includes at least one sleeve of material being semi-transparent to germicidal UV wavelengths and shrunk to said at least one light source to control the output of said at least one light source, said controlled output being limited to levels adequate for microbial growth control.

23. The disinfecting system of claim 22, wherein said at least one shrunk sleeve of material has UVC transmittance in the range of about 10% to 95%.

24. The disinfecting system of claim 1, wherein said limited output inhibits the degradation of fluid-conveying equipment components disposed within the output range of said at least one light source.

25. A disinfecting method, comprising the steps of:
(a) providing at least one light source having output suitable for use as a germicidal agent, said at least one light source being operatively housed in fluid-conveying equipment for fluid disinfection;
(b) limiting the output of said at least one light source to levels adequate for microbial growth control; and
(c) attenuating said limited output by fluid-conveying equipment components disposed within the output range of said at least one light source, said attenuated output providing safe human exposure levels in the vicinity of fluid intake and exhaust portions of the fluid-conveying equipment.

26. The disinfecting method of claim 25, wherein said limited output inhibits the degradation of fluid-conveying equipment components disposed within the output range of said at least one light source.

27. A disinfecting system, comprising:
an electromagnetic energy source; and
a power supply designed to supply current to said electromagnetic energy source such that a lower level of energy than rated by a standard energy source is emitted, by using a near rated cathode current to maintain lamp life, and a percentage of rated lamp current, within HVAC equipment,
wherein energy output is sufficiently controlled to reduce microbial growth and energy output is reduced to prohibit unsafe levels and reduce degradation of HVAC system components.

28. The device of claim 27, wherein said power supply is integral with an assembly, which may be mounted in a remote location, or within an air plenum containing said electromagnetic energy source.

29. A method of disinfecting air, surfaces, fluids and other things utilizing a lower than standard or rated level of electromagnetic energy, comprising:
providing an electromagnetic energy source;
electrically coupling a power supply to said electromagnetic energy source; and
powering said electromagnetic energy source with a percentage of rated lamp current, and near rated cathode current to reduce emitted energy and to enhance the life of said electromagnetic energy source.

30. The method of claim 29, wherein said power supply and said electromagnetic energy source are integrally coupled.

31. A disinfecting system for disinfecting air, surfaces, fluids and other objects, comprising:
an electromagnetic energy source; and
a power supply electrically coupled to said electromagnetic energy source,
wherein electromagnetic energy is emitted at lower than rated levels by supplying said electromagnetic energy source with near rated cathode current and a percentage of rated lamp current,
wherein said emitted energy is sufficiently controlled to reduce microbial growth and otherwise disinfect, and said emitted energy is reduced to prohibit unsafe levels of emitted energy and to reduce degradation of system components.

32. The device of claim 31, wherein said power supply is integral with an assembly, which may be mounted in a remote location, or within an air plenum containing said electromagnetic energy source.

33. A method of supplying lower than rated levels of electromagnetic energy utilizing a rated electromagnetic energy source, comprising:
supplying the electromagnetic energy source with near rated, cathode current to maintain cathode temperature to allow for acceptable thermal characteristics of the electromagnetic energy source; and
supplying the electromagnetic energy source with a fraction of rated, lamp current to allow the electromagnetic energy source to operate at below rated levels and emit lower than rated or maximum levels of electromagnetic energy.

34. A disinfecting system for disinfecting air, surfaces, fluids and other objects, comprising:
an electromagnetic energy source; and
a power supply electrically coupled to said electromagnetic energy source,
wherein electromagnetic energy is emitted at lower than rated levels by supplying said electromagnetic energy source with near rated cathode current and a percentage of rated lamp current.

35. The disinfecting system in accordance with claim 27 wherein lower operating costs are realized.

36. The disinfecting system in accordance with claim 27 wherein the electrical power input is reduced comparable to a system using a conventional full power lamp.

37. The disinfecting system of claim 5, wherein said at least one power supply is adapted to provide a lamp current flowing from a first cathode to a second cathode of said at least one light source, and a cathode current circulating through each of said first and second cathodes, said lamp current being set at a fraction of the nominal lamp current specified for said at least one light source.

38. The disinfecting system of claim 37, wherein said flowing lamp current causes said at least one light source to emit UV (Ultraviolet) energy.

39. The disinfecting system of claim 38, wherein said circulating cathode current maintains adequate heat at said first and second cathodes to ensure stable performance of said at least one light source.
